(19) United States
(12) Patent Application Publication
(10) Pub. No.: US 2025/0384956 A1
(43) Pub. Date: Dec. 18, 2025

(54) System for Automatic Analysis of Image-Informed Gene Expression Data

(71) Applicant: Wisconsin Alumni Research Foundation, Madison, WI (US)

(72) Inventors: Bridget Shields, Madison, WI (US); Chitrasen Mohanty, Salt Lake City, UT (US); Christian Kendziorski, Madison, WI (US)

(21) Appl. No.: 18/743,487

(22) Filed: Jun. 14, 2024

(51) Int. Cl. G16B 25/10; G16B 40/20; G16B 45/00

(52) U.S. Cl. CPC G16B 25/10; G16B 40/20; G16B 45/00

(57) ABSTRACT

An apparatus for diagnosing a disorder or identifying treatment characterizes tissue samples with spatial transcriptomics data and additional cell function data to provide inputs to a machine learning pattern matching algorithm, allowing the sample to be associated with particular treatments or diagnoses or particular examples of other tissue samples from a training set. An associated tool allows the clinician to view both the spatial transcriptomics data and stained image data of a given tissue sample and allows comparison of different tissue samples with respect to gene expression.

### STATEMENT REGARDING FEDERALLY SPONSORED RESEARCH OR DEVELOPMENT

### CROSS REFERENCE TO RELATED APPLICATION

### BACKGROUND OF THE INVENTION

The present invention relates to systems for clinical and computational analyses of biopsy tissue and, in particular, to a system to assist in diagnosis and in identifying treatment options based on expressed genes and expression location information.

Recent developments for measuring gene expressions in tissues promise to improve our understanding of genetic disorders and idiopathic conditions. Of particular interest are spatial transcriptomics platforms which allow monitoring of gene expression in different locations within the tissue at a near cellular level. Such tools can produce large amounts of data covering many different gene expressions at thousands of tissue points.

Despite this improved access to important gene expression data, spatial transcriptomics has had limited success in refining diagnoses or identifying appropriate therapies.

### SUMMARY OF THE INVENTION

The present invention provides a tool that greatly improves the ability of a clinician to make use of the large amounts of data produced by spatial transcriptomics. In one embodiment, the invention augments a transcriptomics platform showing gene expression measurements with a registered image of a stained tissue providing improved insight into the spatial context of the gene expression, for example, according to cell types or other structures within a tissue. In one embodiment, the tool may provide the ability to evaluate and quantify similarities and differences in gene expressions from different tissue samples.

Importantly, in one embodiment, spatial transcriptomics data can be augmented with cell type information, such as cell function, and input into a database that may be used to identify other tissue samples that are highly similar and for which diagnosis or treatment options are known. By exploiting the powerful pattern matching capabilities of machine learning or other computational approaches, the wealth of data obtained from spatial transcriptomics may be harnessed to enhance diagnostic or treatment information, independent of a complete understanding of the underlying cellular mechanisms. Using this information, clinicians may match subsequent and different tissue samples based on clinical information and histologic patterning in a comprehensive way to obtain insights into diagnosis and treatment from existing and trained data.

Specifically then, in one embodiment the invention may provide an apparatus for assessing gene expression in disorders, the apparatus having a first input for receiving first data including spatial transcriptomics data from a biopsy sample and a second input receiving second data including information characterizing cell types of the biopsy sample independent of the spatial transcriptomics data. A machine learning system receives the first and second data and is trained with a training set of multiple training biopsy samples each having corresponding first data and corresponding second data and each linked to a diagnosis or treatment. As so trained, the machine learning system may provide matching tissue samples or output a diagnosis or treatment.

It is thus a feature of at least one embodiment of the invention to use spatial transcriptomics data augmented with other cell data to improve clinical practice in diagnosis and treatment, leveraging machine learning to identify complex linkages between gene expression, location, and cell function that may not be apparent or decipherable by a clinician.

The second data may be stained image data of the biopsy sample.

It is thus a feature of at least one embodiment of the invention to supplement the spatial transcriptomics data with well-characterized stained image information providing spatial transcriptomics data that is likely orthogonal to that obtained from spatial transcriptomics.

The information characterizing cell types may provide a cell type and location for multiple clusters of cells in the biopsy sample.

It is thus a feature of at least one embodiment of the invention to emphasize through clustering important structures in tissue that would be represented by groups of cells in the structure.

The apparatus may further include a segmentation module receiving a stained image of the biopsy sample and deriving cell type and location for multiple clusters.

It is thus a feature of at least one embodiment of the invention to automatically identify functional tissue structures to better inform the machine learning process.

The apparatus may include a data preprocessor receiving the spatial transcriptomics data of the biopsy sample and normalizing the spatial transcriptomics data with respect to the multiple training biopsy samples.

It is thus a feature of at least one embodiment to compensate for differences in biopsy sample size and the like to provide improved comparison with other biopsy samples.

The apparatus may further include a correlator receiving the spatial transcriptomics data to assess cell-to-cell communication within and between clusters and to provide the same to the machine learning system.

It is thus a feature of at least one embodiment of the invention to independently assess cell-to-cell communication as an additional dimension that can be assessed by the machine learning system.

The apparatus may further provide a display displaying information for the biopsy sample together with corresponding information from an identified biopsy sample.

It is thus a feature of at least one embodiment of the invention to allow the identification of specific other biopsy samples through a machine learning process, such other samples which may assist in clinical diagnosis, clinical studies, and the like. This may include the input of tissue, matched to tissue samples included in the original machine learning training, in closest proximity to allow for similarity mapping and subsequent assistance in diagnosis or treatment approach.

These particular objects and advantages may apply to only some embodiments falling within the claims and thus do not define the scope of the invention.

### BRIEF DESCRIPTION OF THE DRAWINGS

FIG. 1 is a block diagram of hardware components suitable for practice of the present invention including an electronic computer implementing multiple software modules for processing spatial transcriptomics and image data;

FIG. 2 is a process block diagram showing processing of the received spatial transcriptomics data and image data and additional clinician input using the hardware and modules of FIG. 1;

FIG. 3 is a representative screenshot provided by the present invention on a display screen showing registered portions of the spatial transcriptomics data and image data; and

FIG. 4 is a representative second screenshot provided by the present invention on a display screen showing differential quantification between two different biopsy samples.

### DETAILED DESCRIPTION OF THE PREFERRED EMBODIMENT

Referring now to FIG. 1, an image-informed diagnostic system 10 according to one embodiment of the present invention may receive image data 12 using an imaging microscope 14, the image data taken of a biopsy sample 16, for example, of skin or other tissue. In one nonlimiting example, the biopsy sample 16 may be a 6-mm punch biopsy stained with hemotoxylin & eosin (H&E) stained images and immediately frozen in OCT media for sectioning and imaging. The image data 12 of a section is then provided to an input of a controller 17 as will be discussed below.

After imaging, the biopsy sample 16 may be subject to analysis by spatial transcriptomics, for example, according to the Visium protocol per the Visium Spatial Gene Expression workflow from 10x Genomics of Pleasanton, California, USA. In this example, the spatial transcriptomics may use a spotted arrays of mRNA-capturing probes on the surface of glass slides. A representative slide may provide for approximately 5000 spatially barcoded spots, which in turn contain millions of spatially barcoded capture oligonucleotides. Each barcoded spot may be 55 μm in diameter, and the distance from the center of one spot to the center of another maybe approximately 100 μm. The spots may be staggered to minimize the distance between them. On average, mRNA from anywhere between 1 and 50 cells are captured per spot which provides near single-cell resolution.

The spatially barcoded, ligated probe products are then released from the slide and sequenced by a sequencer 18. This data provides a list of expressed genes linked to regions of the spots and hence the specific locations that can be registered to the image data 12. The resulting spatial transcriptomics data 20 is also provided to controller 17.

The invention is not limited to this particular form of spatial transcriptomics but may make use of any technique that provides for the identification of expressed genes mapped to particular locations in the tissue with similar resolution.

The invention also employs a training set 22 providing image data 12 and spatial transcriptomics data 20 for many different biopsy samples 14 (not shown) that will be used to train machine learning to be described below. The data of the training set 22 of biopsy samples is contained in a database 26 to be used not only for training but also for library type access of particular biopsy samples 14 and their associated image data 12 and spatial transcriptomics data 20 as will be described. The training set 22 may include formalin-fixed paraffin-embedded tissue, as well as fresh frozen tissue, and the continued enrollment of tissue into the dataset will provide additional training specimens for algorithms developed.

Desirably some or all of the biopsy samples 14 of the library may be linked to a diagnosis or treatment 24 found to be effective for that particular biopsy sample 16. In all cases, the biopsy samples 14 will include other important information, for example, cell type described with respect to organs or cell function, patient biographic information, and the like. The spatial transcriptomics data 20 of the library of biopsy samples 14 will ideally use a similar or identical Visium protocol. This protocol may be performed on previously acquired biopsy samples 14 that may have been preserved with formalin-fixed paraffin-embedding (FFPE) through the steps of: deparaffinizing, staining with H&E and de-crosslinking followed by probe hybridization, probe ligation, and probe release and extension. This protocol may also be performed on prospectively acquired biopsy samples that have been frozen as described previously.

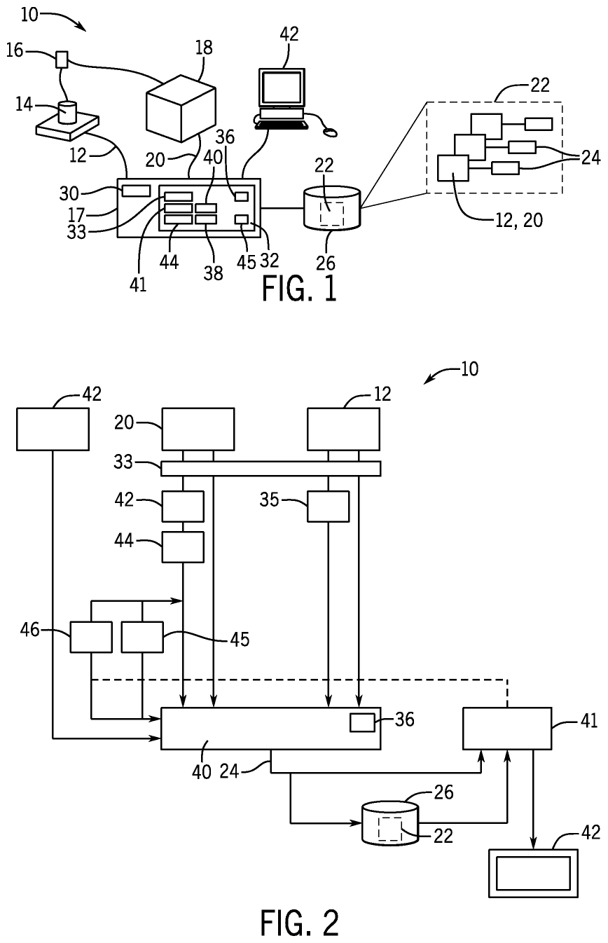

Referring to both FIGS. 1 and 2, the controller 17 may employ a programmable computer or server having one or more processors 30 communicating with electronic memory 32. The electronic memory 32 may include programs implementing a machine learning engine 40 that will be described below programmed with weights 36 derived from the training set 22. Additional programs and electronic memory 32 provide for registration module 33, normalization module 44, segmentation module 35, clustering module 43, and cell-to-cell communication module 38 as will be discussed below. Electronic memory 32 further holds a display module 41 presenting information to a user via a terminal 42, the latter providing, for example, a graphic display, keyboard, mouse, or the like.

Referring now specifically to FIG. 2, the image data 12 and spatial transcriptomics data 20 may be registered to each other by registration module 33 providing them with a common set of location measurements linking pixels of the image data 12 to particular locations of gene expressions of the spatial transcriptomics data 20. This registration process, for example, may be through mechanical registration done at the time of imaging (for example, using a common slide) or through a shift and image correlation process operating on the different data sets.

The spatial transcriptomics data 20 may be provided directly to a machine learning engine 40 and/or optionally also to a clustering module 43, the latter of which performs a clustering according to expressed genes to collect various sample points into a limited number of clusters. The values of the expressed genes (for example, as represented by the number of sample points exhibiting that expression) are then integrated for each cluster and normalized by cluster area per normalization module 44 before being provided as an input to the machine learning engine 40.

The clustered and normalized spatial transcriptomics data may be optionally also provided to a correlation module 45 operating to assess correlations among the genes and/or to a communication module 46 operating to assess cell-to-cell communications based on expressed genes in adjacent spots or spots of interest or among the clusters. This information, linking spots to other spots and a particular cluster to other clusters to which there is substantial correlation between expressed genes provides an additional dimension of understanding of the biopsy sample 16 and, in that respect, may be part of the training set 22 and used by the machine learning engine 40. Techniques for inferring this gene-gene correlations are described in Bernstein et al., 2022, Cell Reports Methods 2, 1 00369 Dec. 19, 2022 https://doi.org/10.1016/j.crmeth.2022.100369, and techniques for inferring cell-to-cell communication are described in Cang, Z., Zhao, Y., Almet, A. A. et al. Screening cell-cell communication in spatial transcriptomics via collective optimal transport. Nat Methods 20, 218-228 (2023). https://doi.org/10.1038/s41592-022-01728-4 both hereby incorporated in its entirety.

Referring still to FIG. 2, the image data 12, which provides pixel intensity values of the stained biopsy sample 16 registered to the spatial transcriptomics data 20 by registration module 33, may then be provided directly to the machine learning engine 40 and/or optionally, in parallel to a segmentation module 35 operating to segment the image data 12 into clusters based on imputed cellular function. This may be done manually or by a machine learning segmentation technique of a type known in the art. Location information about each cluster, for example, a centroid, or centroid plus area data, and the cell type, is then provided to the machine learning engine 40.

Additional information about the biopsy sample 16 may be entered through the terminal 42 by a clinician, for example, patient information such as sex, age, or the like, and the tissue organ source identification that may help inform the analysis process. This information may be provided directly to the machine learning engine 40 and may also be incorporated into the data of the training set 22.

The machine learning engine 40 making use of the weights 36 may then categorize the given biopsy sample with respect to the diagnosis or treatment 24 characterized by the training set 22. This characterization may in turn be used to index through the training set 22 to provide specific examples of biopsy samples 16 that may be similar to the given biopsy samples 16. The additional spatial information provided in the present invention is expected to greatly increase the ability to characterize tissues both with respect to the underlying condition and potential treatments that can, for example, be used to affect particular gene expressions or the like.

The information from the correlation module 45, the communication module 46, the normalization module 44, the segmentation module 35, as well as the spatial transcriptomics data 20 and image data 12, may be provided to a display module 41 aiding the clinician in viewing and understanding the tissue being analyzed. The display module may also receive information related to the desired diagnosis or treatment 24 which may be displayed on the terminal 42 as well as a database of the training set 22 as will be discussed.

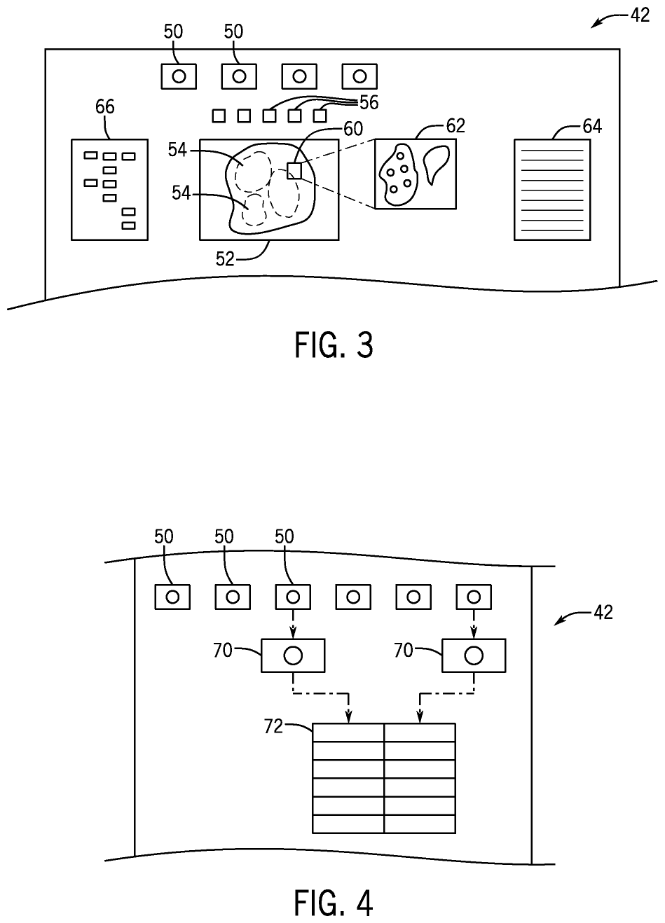

Referring now to FIG. 3, in one mode of operation, the display module 41 may allow for the loading of data associated with one or more biopsy samples 16 having image data 12 displayed as thumbnails 50. A particular thumbnail 50 may then be selected, for example, by a mouse click to display the corresponding spatial transcriptomics data 20 in transcriptomics display area 52 with each sample point indicated by a marker dot and each clusters 54 shaded, for example, in a unique color. These colors are reproduced in corresponding cluster buttons 56 which may be used to select one or more of the clusters 54 for analysis and display, with the clusters 54 not selected having their colors removed.

A cursor box 60 may be movable by the mouse or other cursor control device over the transcriptomics display area 52, the cursor box 60 enclosing a small area that defines a subset of samples of the spatial transcriptomics data 20 within one or more clusters 54. The size of the cursor box 60 may be adjusted by auxiliary controls not shown. The particular region within the cursor box 60 is enlarged in image display 62 adjacent to the transcriptomics display area 52 showing image data 12 directly. This allows the clinician to view the structure of the tissue of the biopsy sample 16 as stained, for example, to identify tissue types. Identified tissue types per this process may be incorporated into training set 22 when the present invention is used to develop training set information.

Expressed genes are displayed for the region of the cursor box 60 in a table list 64 in order of expression amount (number of samples having that expressed gene), and a heat map 66 may also be provided having a vertical axis indicating cluster number and a horizontal axis of gene identifiers with a color mapping to indicate the intensity of the expression. Each small block (rectangle) of the heat map 66 represents a gene. A color intensity of the block shows the expression level of the gene at the location of the cursor box 60. As expressions of multiple genes (˜20,000) are measured at a spot, the heat map 66 shows only selected genes (namely marker genes-algorithmically determined as mentioned: normalization, clustering etc.). The blocks are grouped into clusters in the heat map 66.

In one embodiment, images of biopsy samples 16 associated with the training set 22 identified by the machine learning engine 40 as matching to a current biopsy sample 16 may be used to populate these other boxes to allow manual confirmation of the degree of similarity of these tissues in assessing the diagnosis or treatment suggested.

Referring now to FIG. 4, the display module 41 may alternatively or in addition allow a loading of a set of different biopsy samples 16 (spatial transcriptomics data and image data 12) whose images will be indicated by thumbnails 50′. Particular biopsy sample data from the thumbnails 50′may be selectively dragged into a first or second (or potentially more) comparison group boxes 70 to provide a comparison of gene expressions in these two different biopsy samples 16. This expression may be, for example, by means of a heat map 72 where each row indicates a particular expressed gene (for example, selected according to the first biopsy sample 16) and the first and second columns showing the degree of expression of that gene in the two different samples being compared. This comparison process can be extended to multiple biopsy samples 16.

Generally this comparison process may effect a pseudobulk t-score analysis.

Additional details of the process and materials used in the present invention can be found in Bioinformatics, 2024, 40(3), btae117 https://dolorg/10.1093/bioinformaticsibtaell7 Advance Access Publication Date: 5 Mar. 2024, hereby incorporated in its entirety by reference.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a microprocessor” and “a processor” or “the microprocessor” and “the processor,” can be understood to include one or more microprocessors that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What we claim is:

1. An apparatus for assessing gene expression in disorders comprising:
a first input for receiving first data including spatial transcriptomics data for a biopsy sample providing gene expression data linked to spatial location in the sample;
a second input receiving second data including information characterizing cell types of the biopsy sample independent of the spatial transcriptomics data; and
a machine learning system receiving the first and second data and trained with a training set of multiple training biopsy samples each having corresponding first data and corresponding second data and each linked to a diagnosis or treatment, the machine learning system outputting at least one diagnosis or treatment based on the training set.

2. The apparatus of claim 1 wherein the second data is stained image data of the biopsy sample.

3. The apparatus of claim 2 wherein the stained image data of the multiple training biopsy samples and the biopsy sample are stained with hematoxylin and eosin stain.

4. The apparatus of claim 1 wherein the information characterizing cell types provides a cell type and location for multiple clusters of cells in the biopsy sample.

5. The apparatus of claim 4 further including a segmentation module receiving a stained image of the biopsy sample and deriving cell type and location for multiple clusters of cells in the biopsy sample registered with respect to the spatial transcriptomics data from the stained image.

6. The apparatus of claim 1 further including a data preprocessor receiving the spatial transcriptomics data of the biopsy sample and normalizing the spatial transcriptomics data with respect to the multiple training biopsy samples.

7. The apparatus of claim 6 wherein the data preprocessor further receives the spatial transcriptomics data to collect this into clusters having locations and provides gene expression information to the machine learning system identified to clusters and locations of clusters.

8. The apparatus of claim 1 wherein the information characterizing the cell types of the biopsy sample independent of the spatial transcriptomics data are text descriptions of the biopsy sample by a clinician.

9. The apparatus of claim 1 further including a correlator receiving the spatial transcriptomics data to assess gene-gene correlations and cell-to-cell communication; and
wherein the machine learning system further receives cell-to-cell communication data; and
wherein the training set of multiple training biopsy samples include cell-to-cell communication data.

10. The apparatus of claim 1 wherein the information characterizing cell types of the biopsy sample independent of the spatial transcriptomics data include at least one of organ type and disease type.

11. The apparatus of claim 1 further providing a display displaying the spatial transcriptomics data and information characterizing the cell types of the biopsy sample together with spatial transcriptomics data information characterizing the cell type of at least one training biopsy sample.

12. The apparatus for displaying spatial transcriptomics data comprising:
a first input for receiving first data including spatial transcriptomics data for a biopsy sample providing gene expression data linked to a spatial location in the sample;
a second input receiving second data providing a stained image of the biopsy sample;
a graphic display; and
a display controller executing a program contained in stored memory to:
(1) simultaneously display a spatial representation of the spatial transcriptomics data and the stained image;
(2) provide a cursor operating to identify portions of the spatial representation of the spatial transcriptomics data and stained image having a same location in the biopsy sample; and
(3) provide gene expression data specific to the portion of the spatial representation of the spatial transcriptomics data.

13. The apparatus of claim 12 wherein the spatial representation of the spatial transcriptomics data is collected into clusters based on gene expression and wherein the display controller further receives a cluster selection input to display only selected clusters in the gene expression data specific to the portion of the spatial representation of the spatial transcriptomics data.

14. The apparatus for displaying spatial transcriptomics data comprising:
a first input for receiving first data including spatial transcriptomics data for a first biopsy sample providing gene expression data linked to a spatial location in the sample;
a second input receiving second data indicating spatial transcriptomics data for a second biopsy sample providing gene expression linked to spatial locations in the sample;
a third input for receiving at least one identification of an expressed gene within a set of expressed genes of the first biopsy sample and second biopsy sample;
a graphic display; and
a display controller executing a program contained in stored memory to:
(1) simultaneously display a spatial representation of the spatial transcriptomics data for the first biopsy sample and second biopsy sample; and
(2) display a relative expression of the expressed gene of the third input between the first biopsy sample and second biopsy sample.
